(19) United States
(12) Patent Application Publication
(10) Pub. No.: US 2020/0121712 A1
(43) Pub. Date: Apr. 23, 2020

(54) AGENT TO BE APPLIED TO OPHTHALMIC DEVICE

(71) Applicant: SEIKAGAKU CORPORATION, Tokyo (JP)

(72) Inventor: Keiichiro ARAI, Tokyo (JP)

(73) Assignee: SEIKAGAKU CORPORATION, Tokyo (JP)

(21) Appl. No.: 16/720,909

(22) Filed: Dec. 19, 2019

Related U.S. Application Data
(63) Continuation of application No. 15/753,886, filed on Feb. 20, 2018.

(30) Foreign Application Priority Data
Aug. 20, 2015 (JP) 2015-162578

(51) Int. Cl. A61K 31/728; A61K 9/00; A61P 27/04; A61K 9/08

(52) U.S. Cl. CPC A61K 31/728; A61K 9/08; A61P 27/04; A61K 9/0051

(57) ABSTRACT

Provided is a preparation to be applied to an ophthalmic device comprising a cinnamic acid derivative, the application of which to the ophthalmic device can ameliorate symptoms on the ocular surface or prevent damage on the ocular surface.

### CROSS-REFERENCE TO RELATED APPLICATIONS

This is a continuation application of Ser. No. 15/753,886, filed on Feb. 20, 2018, which is a National Stage Entry of International Patent Application No. PCT/JP2016/074203, filed on Aug. 19, 2016 which claims priority to Japanese Patent Application 2015-162578, filed on Aug. 20, 2015. The disclosure of each of these applications is hereby incorporated by reference in its entirety.

### TECHNICAL FIELD

The present invention relates to a preparation to be applied to an ophthalmic device.

### BACKGROUND ART

A contact lens, an ophthalmic device, brings less distortion and change in size of an image, as compared to those brought by eyeglasses, because of its characteristics in that the distance between a cornea and a contact lens is close to zero. A contact lens is functionally superior to eyeglasses in many points. However, heavy burden is imposed on an eye by wearing of a contact lens, because of direct contact of a contact lens with a cornea. Particularly in a soft contact lens which exhibits bandage effects (lesions are disadvantageously masked/pain is not immediately perceived), corneal disorder may not be recognized until the disorder worsens. Further, when a person inserts a contact lens into his/her eye (especially at the time of the first use of new contact lens), the person may feel uncomfortable because of his/her feeling of a foreign body in his/her eye.

A palpebral conjunctiva has a portion named “lid-wiper” in the vicinity of the lid margin between the sulcus infrapalpebralis and mucocutaneous junction, the portion which may cause friction with the eye surface during a blinking. An epithelial disorder occurred in this portion is called “lid-wiper epitheliopathy (LWE)”. It has been reported that LWE is observed more frequently in a soft contact lens-wearing eye with dry eye symptoms, as compared to a soft contact lens-wearing eye without such symptoms. Therefore, it is fully conceivable that the friction between the lid-wiper and the surface of a soft contact lens may be closely related to the dry eye symptoms of a soft contact lens-wearing eye.

In the meantime, it has been known that a derivative of hyaluronic acid having bonded thereto cinnamic acid (for example, see JP 2009-511423A, hereinafter hyaluronic acid is referred to as “HA”) can be applied to mucous membrane of eyes or the like.

### RELATED ARTS

### Patent Documents

Patent document 1: JP 2009-511423A

### SUMMARY OF THE INVENTION

### Problems to be Solved by the Invention

An object of the present invention is to provide a preparation to be applied to an ophthalmic device the application of which to the ophthalmic device can ameliorate symptoms on the ocular surface or prevent disorders on the ocular surface, and an ophthalmic device coated with the preparation to be applied to the ophthalmic device.

### Means to Solve the Problems

The present inventors have found that by coating an ophthalmic device to be contacted with the ocular surface with a composition containing a cinnamic acid derivative, especially a compound in which a glycosaminoglycan (for example, HA) and a cinnamic acid ester having an amino group are covalently bonded, the ophthalmic device exerts an effect of ameliorating symptoms on the ocular surface and an effect of preventing disorders on the ocular surface. The present invention has been completed, based on this finding.

The present invention includes the following embodiments.

<1> A preparation to be applied to an ophthalmic device, which comprises a cinnamic acid derivative.
<2> The preparation to be applied to an ophthalmic device according to <1>, wherein the cinnamic acid derivative is a compound in which a cinnamic acid ester having an amino group and a glycosaminoglycan are covalently bonded.
<3> The preparation to be applied to an ophthalmic device according to <1> or <2>, wherein the ophthalmic device is applied to the ocular surface
<4> The preparation to be applied to an ophthalmic device according to any one of <1> to <3>, wherein the preparation is at least one of a preparation for immersion, a preparation for coating, a preparation for preservation and a preparation for inhibition of ultraviolet transmission.
<5> A coated ophthalmic device which comprises:

an ophthalmic device, and

the preparation to be applied to the ophthalmic device of any one of <1> to <4>, the preparation coating the ophthalmic device.

<6> A device for treating diseases of the ocular surface, which comprises the coated ophthalmic device of <5>.
<7> Use of a cinnamic acid derivative for the manufacture of a preparation to be applied to an ophthalmic device.
<8> Use of a cinnamic acid derivative for the manufacture of a device for treating diseases of the ocular surface.
<9> A method for treating diseases of the ocular surface, which comprises putting the device of <5> on the ocular surface.
<10> An ophthalmic device-containing article, which comprises:

at least one ophthalmic device, and

a container containing the ophthalmic device,

wherein the ophthalmic device is immersed in the preparation to be applied to an ophthalmic device of any one of <1> to <4>.

### Effects of the Invention

According to the present invention, it is possible to provide a preparation to be applied to an ophthalmic device, the application of which to the ophthalmic device can ameliorate symptoms on the ocular surface or prevent disorders on the ocular surface, an ophthalmic device coated with the preparation to be applied to an ophthalmic device, a device for treating diseases of the ocular surface, and the like.

### BRIEF DESCRIPTION OF THE DRAWING

FIG. 1 shows the degree of staining with fluorescein when the preparation according to the present embodiment to be applied to an ophthalmic device is administered once a day to an eye of a model animal.

### MODES FOR CARRYING OUT THE INVENTION

Hereinbelow, the embodiments of the present invention will be described. In the present specification, with respect to each component in a composition, unless otherwise stated, when a plurality of substances belonging to the component are present in the composition, the content of the component means the total amount of the substances present in the composition. The term “process” is not limited to an independent and this term includes any process as long as the intended purpose of the process is achieved, even if the process cannot be clearly distinguished from other processes.

(1) Preparation to be Applied to an Ophthalmic Device

The preparation to be applied to an ophthalmic device contains a cinnamic acid derivative.

(1-1) Cinnamic Acid Derivative

There is no particular limitation with respect to the cinnamic acid derivative contained in the preparation to be applied to an ophthalmic device, as long as the derivative is a compound having, as a part of its structure, a structure derived from cinnamic acid. As examples of the cinnamic acid derivatives, there can be mentioned a derivative in which the carboxyl group of cinnamic acid forms an ester bond or amide bond to have a substituent; a derivative in which the phenyl group of cinnamic acid is substituted with 1 to 5 substituent(s); and a derivative having a substituent on both of the carboxyl group and phenyl group. Among these derivatives, preferred is a cinnamic acid ester in which the carboxyl group of cinnamic acid forms an ester bond to have a substituent, and particularly preferred is a derivative in which a cinnamic acid ester having an amino group and a glycosaminoglycan (hereinafter abbreviated as “GAG”) are covalently bonded. As examples of the “cinnamic acid ester having an amino group”, there can be mentioned a cinnamic acid aminoalkyl ester, a cinnamic acid aminoalkenyl ester, a cinnamic acid aminoalkynyl ester and the like.

The “alkyl” moiety of the above-mentioned “cinnamic acid aminoalkyl ester” is not limited to a linear one, and the methylene group constituting the “alkyl” moiety may have a substituent, such as an alkyl group, an aryl group, a hydroxyl group, a halogen atom and the like. Similarly, each of the “alkenyl” moiety of the “cinnamic acid aminoalkenyl ester” and the “alkynyl” moiety of the “cinnamic acid aminoalkynyl ester” is not limited to a linear one and may have a similar substituent.

In this connection, as examples of the carbon number of the main chain of the “alkyl” moiety of the “cinnamic acid aminoalkyl ester”, there can be mentioned 1 to 18, 1 to 12, 1 to 6 and 2 to 3, and 2 to 3 is preferred. As examples of the carbon number of the main chain of each of the “alkenyl” of the “cinnamic acid aminoalkenyl ester” and the “alkynyl” of the “cinnamic acid aminoalkynyl ester”, there can be mentioned 2 to 18, 2 to 12, 2 to 6 and 2 to 3, and 2 to 3 is preferred.

As specific examples of the “cinnamic acid aminoalkyl ester”, there can be mentioned cinnamic acid aminoethyl ester, cinnamic acid aminopropyl ester and the like. Among these, preferred is at least one of cinnamic acid 2-aminoethyl ester and cinnamic acid 3-aminopropyl ester, and particularly preferred is cinnamic acid 3-aminopropyl ester.

It should be readily understood that in the present specification including the following explanation, the term “cinnamic acid ester having an amino group” encompasses, and may be replaced with, these specific or preferred cinnamic acid esters.

A GAG to which such a “cinnamic acid ester having an amino group” is covalently bonded is an acidic polysaccharide having a structure composed of disaccharide repeating units each consisting of an amino sugar and uronic acid (or galactose). As examples of such GAGs, there can be mentioned HA, chondroitin, chondroitin sulfate, dermatan sulfate and the like and, among these, HA is preferred. There is no particular limitation with respect to HA, as long as the constitutional unit of HA is a disaccharide unit consisting of N-acetyl-D-glucosamine and D-glucuronic acid bonded through a β-1,3 bond and HA is constituted with a plurality of the disaccharide units repeatedly bonded through β-1,4 bonds. The GAG may be in a free form in which no salt is formed or form a pharmaceutically acceptable salt.

Examples of pharmaceutically acceptable salts of the GAG include a salt with an alkali metal ion, such as a sodium salt and a potassium salt; a salt with an alkaline earth metal ion, such as a magnesium salt and a calcium salt; a salt with an inorganic base, such as an ammonium salt; a salt with an organic base, such as diethanolamine, cyclohexylamine, an amino acid and the like. For example, as a pharmaceutically acceptable salt of HA, a salt with an alkali metal ion is more preferred, and a sodium salt is particularly preferred.

The GAG can be produced by a conventionally known method, depending on its type. As examples of such methods, there can be mentioned extraction and purification from animal-derived materials, culture and purification from GAG-producing bacteria or the like, sugar chain modification, sugar chain synthesis and the like.

Specifically, with respect to HA, HA may either be derived from a natural product obtained by extracting a part of a living body, such as cockscomb, umbilical cord, cartilage, skin or the like; chemically synthesized; or produced by culturing microorganisms or genetic engineering technique. The preparation to be applied to an ophthalmic device is applied to an existing ophthalmic device which is contacted with the ocular surface. Therefore, preferred is highly pure HA which contains substantially no substance which is unpermitted to contaminate a pharmaceutical.

There is no particular limitation with respect to the weight average molecular weight of a GAG and, as examples of that for HA, there can be mentioned 10,000 to 5,000,000, preferably 200,000 to 3,000,000, more preferably 500,000 to 2,500,000. The weight average molecular weight of HA can be determined by the intrinsic viscosity method. With respect to chondroitin or chondroitin sulfate, the weight average molecular weight is preferably 10,000 to 200,000, more preferably 10,000 to 60,000. The weight average molecular weight of chondroitin and chondroitin sulfate can be determined by size exclusion chromatography or a light scattering method.

The compound in which a cinnamic acid ester having an amino group and a GAG are covalently bonded can be obtained by covalently bonding such a GAG and a cinnamic acid ester having an amino group. There is no particular limitation with respect to the manner of this covalent bond, and it is preferred that by the covalent bond, an amino group of the cinnamic acid ester and a carboxyl group of the GAG are bonded through an amide bond. Hereinbelow, explanation is made with reference to the cinnamic acid derivative in which the cinnamic acid ester and HA are bonded through an amide bond (hereinafter referred to as “HA-cinnamic acid derivative”) cited as an example.

In the HA-cinnamic acid derivative, the amide bond has to bond not all carboxyl groups but at least a part of the carboxyl groups of the HA to the cinnamic acid ester having an amino group. Hereinbelow, the ratio of the amount of carboxyl groups forming the amide bonds in the GAG, relative to the total amount of carboxyl groups present in the GAG, is referred to as the “degree of substitution (DS)”. DS is calculated as the ratio (%) of introduction of residues of the cinnamic acid ester having an amino group, based on the number of disaccharide units constituting the HA. For example, with respect to the HA derivative having introduced thereinto residues of the cinnamic acid ester having an amino group, when the ratio of introduction of residues is 1 residue per 1 disaccharide unit or 1 residue per 200 monosaccharide residues (100 units in terms of the number of the disaccharide units), DS is 100% or 1%, respectively.

With respect to the HA-cinnamic acid derivative, as an example of preferred DS, there can be mentioned 3 to 50%. DS is preferably 3 to 30%, more preferably 10 to 20%, and still more preferably 12 to 18%.

The HA-cinnamic acid derivative can be produced by, for example, bonding through amide bonds an amino group, which is derived from an aminoalkanol (for example, an aminoethanol (such as 2-aminoethanol) or an aminopropanol (such as 3-aminopropanol)) constituting the cinnamic acid aminoalkyl ester, with the carboxyl groups of the HA.

Such a cinnamic acid aminoalkyl ester is an ester compound in which the carboxyl group of the cinnamic acid and the hydroxyl group of an aminoalkanol form an ester bond. As described above, the cinnamic acid constituting the cinnamic acid aminoalkyl ester may be a substituted cinnamic acid in which the phenyl group is substituted with 1 to 5 substituents.

A preferred structure of the HA-cinnamic acid derivative can be represented by the following general formula (I):

[Ar—CH═CH—COO—(CH₂)ₙ—NH—]ₘ—HA′ (I)

wherein Ar represents a phenyl group which may have a substituent; n represents 2 or 3; HA′ represents a carboxy residue of the HA; m represents the ratio of amidated carboxyl groups among all carboxyl groups in the HA, which is 3 to 50% relative to the total number of carboxyl groups.

The HA-cinnamic acid derivative can be produced in accordance with the method described in, for example, JP 2002-249501A, WO 2008/069348 or the like. Specifically, there is no particular limitation with respect to such a method as long as a cinnamic acid aminoalkyl ester can be chemically bonded to HA through amide bonds by the method. As examples of such methods, there can be mentioned a method using a water-soluble condensation agent, such as a water-soluble carbodiimide (for example, 1-ethyl-3-(3-dimethylaminopropyl)carbodiimide hydrochloride (EDCI.HCl), 1-cyclohexyl-3-(2-morpholinoethyl)carbodiimide-meth-p-toluene sulfonate); a method in which a condensation aid, such as N-hydroxysuccinimide (HOSu) and N-hydroxybenzotriazole (HOBt), is used in combination with the above-mentioned condensation agent; a method using a condensation agent, such as 4-(4,6-dimethoxy-1,3,5-triazin-2-yl)-4-methylmorpholinium chloride (DMT-MM); an active ester method; an acid anhydride method and the like.

The HA-cinnamic acid derivative may be prepared by a method in which cinnamic acid is preliminarily reacted with an aminoalkanol (such as 3-aminopropanol (this applies to the same term hereinafter appearing)) to prepare a cinnamic acid aminoalkyl ester (such as cinnamic acid 3-aminopropyl ester (this applies to the same term hereinafter appearing)) and the amino group of the resultant cinnamic acid aminoalkyl ester is bonded to the carboxyl groups of the HA through amide bonds; or a method in which the amino group of an aminoalkanol is bonded to the carboxyl groups of the HA through amide bonds to prepare an HA having introduced thereinto the aminoalkanol and, thereafter, the carboxyl group of cinnamic acid is bonded to the hydroxyl groups (derived from the aminoalkanol) of the HA having introduced thereinto the aminoalkanol through ester bonds.

The cinnamic acid derivative can be used for producing a preparation to be applied to an ophthalmic device or a below-mentioned device for treating diseases of the ocular surface.

(1-2) Ophthalmic Device

There is no particular limitation with respect to the ophthalmic device to which the preparation to be applied to an ophthalmic device is applied, as long as the device is a medical device used in the field of ophthalmology, and it is preferred that the device is applied to the ocular surface. As examples of devices applied to the ocular surface, there can be mentioned a contact lens, a scleral depressor, an eye speculum, a lens hook and ophthalmic scissors and the like. Among them, it is preferred that the preparation is applied to a contact lens. Further, there is no particular limitation with respect to the type of the contact lens to which the preparation is applied and any type can be used. As examples of the types, there can be mentioned a soft contact lens, a hard contact lens, a disposable contact lens, an oxygen-permeable contact lens, a color contact lens and the like.

(1-3) Use

There is no particular limitation with respect to the use of the preparation to be applied to an ophthalmic device, as long as the preparation is applied to an ophthalmic device, and the preparation can be for use as, for example, a preparation for immersion, a preparation for coating, a preparation for preservation, a preparation for inhibition of ultraviolet transmission and the like. When the preparation to be applied to an ophthalmic device is used as a preparation for immersion, the preparation may be used for immersing the ophthalmic device entirely or immersing only a portion of the device to contact the ocular surface.

For example, when the preparation to be applied to an ophthalmic device is used as a preparation for immersion of a contact lens, the contact lens may be immersed in the preparation in a manner wherein the preparation is put in a container for the contact lens together with the contact lens, or may be immersed in the preparation just before the insertion of the contact lens. Further, for example, when the preparation is used as a preparation for immersion of an eye speculum, the eye speculum may be used after only a portion of the eye speculum to contact the ocular surface is immersed before use.

As understood from Examples described later, a corneal epithelial disorder is ameliorated by instilling the preparation to be applied to an ophthalmic device once a day. Therefore, by preliminarily immersing the device to be applied to the ocular surface in the preparation to be applied to an ophthalmic device, a disease of the ocular surface can be expected to be ameliorated when the ophthalmic device contacts the ocular surface.

Further, as shown in Examples described later, the preparation to be applied to an ophthalmic device exhibits, when mixed with mucin contained in tear fluid, high rate of viscosity increase whereby friction in the eye can be reduced. Therefore, in substantially the same manner as above, an effect for preventing a corneal epithelial disorder can be expected to be exhibited when the ophthalmic device contacts the ocular surface. Amelioration of dry eye symptoms in a contact lens-wearing eye can also be expected. Further, by the contact of the preparation to be applied to an ophthalmic device with the ocular surface during use of the ophthalmic device, drying of the ocular surface can also be prevented.

The time of immersion of the ophthalmic device varies depending on the material of the ophthalmic device. For example, if the ophthalmic device itself contains water (like a soft contact lens), the device is immersed for 30 minutes or more. By this treatment, the cinnamic acid derivative can be expected to penetrate inside of the ophthalmic device, be gradually released on the ocular surface and exhibit higher effect for ameliorating a corneal epithelial disorder. Further, by this treatment, it also becomes possible to save effort, such as, in the case of an eye drop, instillation when needed.

When the preparation to be applied to an ophthalmic device is used as a preparation for coating an ophthalmic device, there is no particular limitation with respect to the method for coating the device, as long as a portion of the ophthalmic device to contact the ocular surface is covered with the preparation to be applied to an ophthalmic device. An ophthalmic device can be coated with the preparation to be applied to the device by, for example, spraying, dropping, application, curtain coating or the like with the preparation, or immersing the device in the preparation.

When the ophthalmic device is a contact lens, coating may be achieved also by instilling the preparation to be applied to an ophthalmic device to an eye having inserted thereinto the contact lens. The effects as a preparation for coating the ophthalmic device are substantially the same as those for the above-mentioned preparation for immersion.

When the preparation to be applied to an ophthalmic device is used as a preparation for preservation, there is no particular limitation with respect to the preservation method, as long as the ophthalmic device is preserved in a manner wherein a portion of the device to contact the ocular surface is covered with the preparation to be applied to an ophthalmic device.

As specific examples of methods for utilizing the preservation solution, there can be mentioned, in addition to a method in which the solution is used as a preservation solution to be contained in the below-mentioned ophthalmic device-containing article together with the device, a method in which the solution is used as a preservation solution for an ophthalmic device which can be is preserved in a conventional solution. The effects expected as a preservation solution for the ophthalmic device are substantially the same as those for the above-mentioned preparation for immersion.

Further, since the preparation to be applied to an ophthalmic device contains a cinnamic acid derivative, the preparation can absorb light having a wavelength of 320 nm or less. For example, when a solution (0.1 w/v %) of the cinnamic acid derivative comprising HA having introduced thereinto aminopropyl cinnamate with a degree of substitution of 16% is prepared and ultraviolet transmittance of the solution is measured by a spectrometer (UV-1600, manufactured by Shimadzu Corporation), the solution is shown to be able to absorb 80% or more of light having a wavelength of 320 nm or less. The preparation to be applied to an ophthalmic device can be used as a preparation for inhibition of ultraviolet transmission by applying the preparation to an ophthalmic device in substantially the same manner as that for the above-mentioned preparation for coating. For example, by coating a contact lens with a preparation to be applied to an ophthalmic device, the amount of ultraviolet rays reaching the ocular surface while the contact lens is inserted can be reduced and a corneal epithelial disorder caused by ultraviolet rays can be prevented.

The preparation to be applied to an ophthalmic device may be in any form as long as the preparation is in a solution form when applied to an ophthalmic device, and it is also possible to use a powder of the cinnamic acid derivative dissolved in a solvent at the time of applying to the ophthalmic device.

It is preferred that the pH of the preparation to be applied to an ophthalmic device is adjusted to around 4 to 9, or 5 to 8. When the pH is 4 or more or 9 or less, the eye irritation at the time of contact of the ophthalmic device with the ocular surface is reduced and adverse effects on the material of the ophthalmic device may be suppressed.

A solution of the cinnamic acid derivative can be directly used as the preparation to be applied to an ophthalmic device. However, the preparation may contain, depending on its use, an additive which can be used in the field of a preservation/washing solution for an ophthalmic device or ophthalmology, as long as the effect of the present invention can be exerted. Examples of additives include antiphlogistics, such as allantoin and azulenesulfonic acid; decongestants, such as naphazoline hydrochloride and naphazoline nitrate; antiallergic agents, such as chlorpheniramine maleate and diphenhydramine hydrochloride; algefacients, such as menthol; antibacterial, bactericidal and antiseptic agents, such as polyhexamethylene biguanide and benzalkonium chloride; surfactants, such as polyoxyethylene sorbitan monooleate, polyoxyl 40 stearate and polyoxyethylene hydrogenated castor oil; polyhydric alcohols, such as glycerin, propylene glycol and polyethylene glycol; buffer agents, such as sodium phosphate, sodium hydrogen phosphate, sodium dihydrogen phosphate, sodium acetate and epsilon-aminocaproic acid; tonicity agents, such as sodium chloride, potassium chloride and concentrated glycerin; stabilizing agents, such as sodium edetate; proteolytic enzymes; and lipolytic enzymes. Any additive selected from the above may be contained in the preparation as required.

The preparation to be applied to an ophthalmic device can be used also for producing an ophthalmic device as described later or producing a device for treating diseases of the ocular surface comprising the ophthalmic device.

(2) Coated Ophthalmic Device and Device for Treating Diseases of the Ocular Surface, the Device Comprising the Coated Ophthalmic Device

The coated ophthalmic device is the ophthalmic device as exemplified in (1-2) coated with the preparation to be applied to an ophthalmic device. There is no particular limitation with respect to the method for coating, and the ophthalmic device as exemplified in (1-2) can be coated with the preparation to be applied to the device by spraying, dropping, application, curtain coating or the like with the preparation, or immersing the device in the preparation.

The coated ophthalmic device can be used also as a device for treating diseases of the ocular surface. As a method of treating diseases of the ocular surface, there can be mentioned a method in which the treatment is carried out by contacting the ocular surface with the coated ophthalmic device and, for example, when the device is a contact lens, the ocular surface can be treated by inserting the contact lens.

In the present specification, with respect to the “contact with the ocular surface”, there is no particular limitation for the conditions for or time of the contact, as long as the contact with the ocular surface occurs. For example, the contact may occur over the time required in a surgery, like that of a scleral depressor, and the contact with the ocular surface may occur all day long, like that of a contact lens. When the coated ophthalmic device is a coated contact lens, feeling of a foreign body or uncomfortable feeling at the time of the first insertion of the lens into an eye can be reduced.

In the present specification, the term “ocular surface” means sclera, conjunctiva and cornea. Among these, cornea is preferred, and corneal epithelium is particularly preferred.

In the present specification, the term “treatment” means any treatment to be applied for a disease and, as examples of such treatments, there can be mentioned remedial treatment, amelioration and suppression of progression (prevention of deterioration) of a disease and prevention of disorder.

In the present specification, the term “disease of the ocular surface” means any lesion or other abnormality occurring on the ocular surface, and examples of such diseases include corneal epithelial disorders, such as corneal epithelial defect, corneal epithelial erosion, corneal ulcer, corneal perforation, keratoconjunctivitis and superficial punctate keratitis (SPK). Further, the disease of the ocular surface include corneal epithelial disorders associated with an endogenous disease, such as dry eye, Sjögren's syndrome and Stevens-Johnson syndrome, or corneal epithelial disorders associated with an exogenous disease related to insertion of a contact lens, external injury, surgery, infection, drug or the like.

(3) Ophthalmic Device-Containing Article

The ophthalmic device-containing article comprises a container containing at least one ophthalmic device, the device being immersed in the preparation to be applied to an ophthalmic device. That is, the ophthalmic device-containing article comprises at least one ophthalmic device and a container containing the ophthalmic device, the device being immersed in the preparation to be applied to an ophthalmic device.

The ophthalmic device-containing article can be produced by, for example, the following production method.

A production method comprising the steps of:

putting, in a hermetically sealable container, at least one ophthalmic device together with a preparation to be applied to an ophthalmic device;

hermetically sealing the container with the at least one ophthalmic device immersed in the preparation to be applied to an ophthalmic device; and

subjecting the container to autoclave sterilization.

With respect to the ophthalmic device-containing article of the present invention, preferred embodiments of and conditions for the “ophthalmic device” and the “preparation to be applied to an ophthalmic device” are substantially the same as those described above in (1) preparation to be applied to ophthalmic device and (1-2) ophthalmic device.

### EXAMPLES

Hereinbelow, the present invention will be described with reference to the following Examples. However, they do not limit the technical scope of the present invention.

### [Example 1] Preparation of HA Having Covalently Bonded Thereto a Cinnamic Acid Aminoalkyl Ester

HA having covalently bonded thereto a cinnamic acid aminopropyl ester was prepared in accordance with the method described in Example 2 of JP 2002-249501A using, as a starting material, HA having a weight average molecular weight of 880,000 (measured by intrinsic viscosity method). Hereinbelow, this “HA having covalently bonded thereto a cinnamic acid aminopropyl ester” is abbreviated as “HA-3APC”. As a result of analysis of the produced HA-3APC by the method described in Examples of JP 2002-249501A, the degree of substitution with the cinnamic acid aminopropyl ester, based on the number of disaccharide repeating units constituting HA, was 15.3%.

### [Example 2] Preparation of Eye Drop

Phosphate buffered saline (PBS) was added to HA-3APC (test substance) prepared in Example 1 to thereby prepare solutions (0.5 w/v %, 0.3 w/v % and 0.1 w/v %) of HA-3APC. Then, each of these solutions was subjected to filtration sterilization with a 0.22 μm filter to thereby prepare eye drops. Hereinbelow, these eye drops are referred to as the “0.5% test substance solution”, “0.3% test substance solution” and “0.1% test substance solution”.

### [Example 3] Test Using Disease Animal Model of Dry Eye

(1) Preparation of Animal Model

Male SD rats of 7 weeks old (SPF) were lightly anesthetized with diethyl ether and, under anesthesia with inhalation of isoflurane, the right and left cheeks of the rats were sheared.

The sheared portions were disinfected with a 70% aqueous solution of ethanol, incision of about 7 mm in the longitudinal direction was made at the position about 7 mm below each ear, and the extraorbital lacrimal glands of both eyes were excised. Then, an antibacterial drug (Taribid (registered trademark) ophthalmic ointment) was applied to the incisions, the incisions were sutured, and the sutured cites were disinfected with a 10% povidone-iodine solution.

Two months after the excision of the lacrimal glands, the corneal epithelium of each eye was stained with fluorescein using Flores (registered trademark) test paper (Showa Yakuhin kako Co., Ltd.) under anesthesia with inhalation of isoflurane. By this treatment, the sites of defect (disorder) of the corneal epithelium were stained with fluorescein.

Under lighting with a slit lamp (SL-D7, manufactured by TOPCON CORPORATION), the entire cornea was visually divided into three areas including the upper to lower areas and, with respect to each area, the severity of the corneal epithelial disorder was scored in accordance with the following criteria (the maximum score for one whole eye is 9). The score of each individual was indicated by the average value of both eyes. The scored cornea of each individual was entirely photographed using a digital photographing unit.

(Criteria)
0 point: No dot-like stained site
1 point: Sparse (dot-like fluorescein-stained sites are apart from each other)
2 points: Middle (intermediate state between sparse and dense)
3 points: Dense (most of adjacent dot-like fluorescein-stained sites are in contact with each other)

(2) Grouping The animal models prepared and scored as described above were divided into five groups shown in Table 1 so that the average scores of these groups were the same.

TABLE 1

| Name of | Administered | Number of | Instillation |
|---|---|---|---|
| group | substance | animals (eyes) | volume |
| Control | PBS | 5 Rats | Once: 5 μL |
| group | | (10 eyes) | |
| 0.1% Group | 0.1% Test substance | 5 Rats | Once: 5 μL |
| | solution | (10 eyes) | |
| 0.3% Group | 0.3% Test substance | 5 Rats | Once: 5 μL |
| | solution | (10 eyes) | |
| 0.5% Group | 0.5% Test substance | 5 Rats | Once: 5 μL |
| | solution | (10 eyes) | |
| Normal | None | 3 Rats | — |
| | | (6 eyes) | |

(3) Test Method

With respect to each group, the administered substance according to Table 1 was administered. Instillations were carried out once a day continuously for 21 days (3 weeks) in total, using a continuous dispenser (Multipette Plus, manufactured by Eppendorf). The severity of corneal epithelial disorder was evaluated immediately after the start of administration (0 week) and at 3 days, 1 week, 2 weeks and 3 weeks after the start of administration. Evaluation was carried out by blind scoring in accordance with the criteria described above.

With respect to each group, the results are shown as the mean value±standard error. With respect to each group to which the test substance solution was administered, the dose responsibility of the scores of the group at each point of time for evaluation was confirmed by Shirley-Williams test and Jonckheere-Terpstra test. In each case, a significance level of less than 5% was regarded as significant.

(4) Test Results

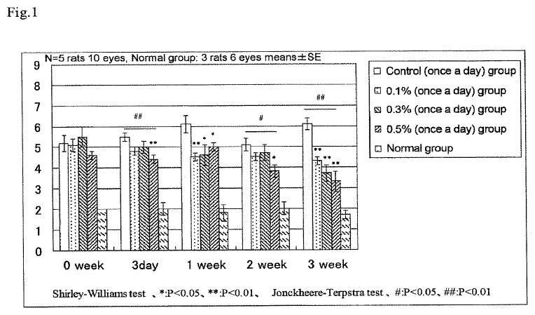

The results are shown in FIG. 1. In the test of the dose response by the Shirley-Williams test, the dose responsibilities were significant against the control group, at all points of time for evaluation from 3 days after the start of administration with respect to the group to which the 0.5% test substance solution was administered, and at 1 week and 3 weeks after the start of administration with respect to each of the group to which the 0.1% test substance solution was administered and the group to which the 0.3% test substance solution was administered.

Further, in the test of trend in the dose responsibility by the Jonckheere-Terpstra test, the Jonckheere statistics at 3 days, 2 weeks and 3 weeks after the start of administration were significant.

The above result shows that application of the preparation of the present invention to be applied to an ophthalmic device once a day significantly ameliorates corneal epithelial disorder. Therefore, corneal epithelial damage can be significantly ameliorated by, for example, wearing a contact lens coated with the preparation of the present invention to be applied to an ophthalmic device. Further, application of the preparation is effective even once a day. Therefore, for example, when the preparation of the present invention to be applied to an ophthalmic device is used as a solution for preservation of disposable a contact lens for one-day use, use of this contact lens can be expected to bring substantially the same result as those brought by application of the preparation to the ocular surface once a day.

### [Example 4] Preparation of the Preparation to be Applied to an Ophthalmic Device

HA-3APC prepared in Example 1 was added a base material (0.7 w/v % sodium chloride and 0.2 w/v % potassium chloride as tonicity agents, 0.03 w/v % sodium hydrogen phosphate and 0.07 w/v % sodium dihydrogen phosphate as buffering agents, 0.1 w/v % disodium edetate as a stabilizer and 0.003 w/v % % benzalkonium chloride as an antiseptic agent), and pH of the resultant mixture was adjusted to be 5.0 to 6.0 to thereby obtain aqueous solutions (concentrations: 0.1 w/v %, 0.3 w/v % and 0.5 w/v %) of HA-3APC. Each of the resultant solutions was subjected to filtration sterilization, and each of the resultant filtrates used as a preparation to be applied to an ophthalmic device.

### [Example 5] Substantially the Same Preparation Procedure as in Example 4 was Repeated, Except that the Base Material was Replaced with Those Given in Table 2

The concentration of glycerin was 0.5 w/v %, and the concentrations of the other components were the same as those in Example 4.

TABLE 2

| | | Base material 1 | Base material 2 | Base material 3 |
|---|---|---|---|---|
| Formulated | Tonicity | Sodium chloride | Sodium chloride | Sodium chloride |
| component | agents | Potassium chloride | Potassium chloride | Potassium chloride |
| | Buffering | Sodium hydrogen | Sodium hydrogen | Sodium hydrogen |
| | agents | phosphate | phosphate | phosphate |
| | | Sodium dihydrogen | Sodium dihydrogen | Sodium dihydrogen |
| | | phosphate | phosphate | phosphate |
| | Stabilizer | — | — | Sodium edetate |
| | Antiseptic | — | — | Benzalkonium |
| | agent | | | chloride |
| | Polyhydric | — | Glycerin | Glycerin |
| | alcohol | | | |

### [Example 6] Viscosity Increasing Effect of Mucin by Interaction

In general, with respect to a film of a lubricant formed on a friction surface for reducing friction under the maintained state of fluid friction, the higher the viscosity of the lubricant, the higher the stability of the film of the lubricant. Therefore, the friction reducing effect of the cinnamic acid derivative was evaluated using, as an index, the increase in viscosity due to the interaction of the derivative with mucin contained in the tear fluid.

HA-3APC prepared in Example 1 was dissolved in PBS (pH 7.4) to thereby prepare solutions having concentrations of 1 w/v %, 0.5 w/v % and 0.25 w/v %.

Similarly, HA was dissolved in PBS (pH 7.4) to thereby prepare an HA solution having a concentration of 1 w/v %.

0.4 mL of each of these samples (1 w/v % HA-3APC, 0.5 w/v % HA-3APC, 0.25 w/v % HA-3APC and 1 w/v % HA) having added thereto 0.4 mL of 20 w/v % mucin was sample 1 for measurement.

A sample of 0.4 mL of 20 w/v % mucin having added thereto 0.4 mL of PBS was sample 2 for measurement.

A sample prepared in substantially the same manner as in sample 1 for measurement except that 0.4 mL of PBS was added in place of mucin was sample 3 for measurement.

Each sample for measurement was subjected, immediately after thorough mixing, to measurement of viscosity (shear rate: 100 S⁻¹, temperature: 35° C.) using a rotatory viscometer (ADVANCED RHEOMETER (TA Instruments)), and increase in value of viscosity was obtained by the following formula.

Increase in value of viscosity=μ1−μ2−μ3

μ1: Viscosity of sample 1 for measurement

μ2: Viscosity of sample 2 for measurement

μ3: Viscosity of sample 3 for measurement

Comparison of the strength of the interaction with mucin was carried out between HA and HA-3APC based on the standardized rate of increase in viscosity obtained by dividing the increase in value of viscosity with the viscosity of sample 3 for measurement. Such a means for standardization is frequently used in comparison of interactions with mucin among different test substances (for example, J. Ocul Pharmacol Ther. 2007.23(6): 541-50).

Increase in value of viscosity was calculated with respect to each mixture obtained by mixing HA-3APC (with concentrations of 0.25 w/v %, 0.5 w/v % and 1.0 w/v %) with a 20 w/v % mucin solution (final HA-3APC concentrations of 0.125 w/v %, 0.25 w/v % and 0.5 w/v %, respectively). The results showed that increase in value of viscosity (indicating the strength of interaction with mucin) increased depending on the concentration of HA-3APC.

Further, the rate of increase in viscosity was calculated with respect to a mixture obtained by mixing HA-3APC (with concentration of 1.0 w/v %) with a 20 w/v % mucin solution (final HA-3APC concentration of 0.5 w/v %) and, similarly, the rate of increase in viscosity was calculated with respect to a mixture obtained by mixing HA with a 20 w/v % mucin solution (with the same concentration as above for HA-3APC). As a result, HA-3APC showed the rate of increase in viscosity about 1.5 times higher than that of HA (1.0 w/v % HA (final concentration 0.5 w/v %): 2.49, 1.0 w/v % HA-3APC (final concentration 0.5 w/v %): 3.65).

These results suggest that the cinnamic acid derivative interacts with mucin in the tear fluid on the ocular surface to have high rate of increase in viscosity. Further, it is conceivable that the friction by the lid-wiper is reduced, and amelioration of dry eye symptoms in eyes wearing a contact lens can also be expected.

### [Example 7] Production of a Coated Ophthalmic Device

A contact lens (one each of A: One Day Acuvue (registered trademark) Moist (registered trademark): Johnson & Johnson Company and B: One Day Pure (registered trademark): Seed Co., Ltd.) was immersed in 5 mL of the preparation to be applied to an ophthalmic device prepared in Example 4 at 24° C. for 5 hours to thereby prepare coated ophthalmic devices.

### [Example 8] Effects on Lens Size of the Preparation to be Applied to an Ophthalmic Device

The contact lenses (the two types described in Example 7) were immersed in the preparation to be applied to an ophthalmic device (using 0.3 w/v % HA-3APC aqueous solution) prepared in Example 4 under the same conditions as those in Example 7, and the lens sizes before and after the immersion were measured. The results are shown in Table 3. These results showed that the present preparation had almost no influence on the lens size.

TABLE 3

| | Before immersion | After immersion | Change in value |
|---|---|---|---|
| | (mm) | (mm) | (mm) |
| Contact lens A | 13.86 | 13.70 | 0.16 |
| | 13.98 | 13.94 | 0.04 |
| Contact lens B | 14.03 | 13.94 | 0.09 |
| | 14.08 | 14.06 | 0.02 |

Disclosures of Japanese Patent Application No. 2001-385072 (filing date: Dec. 18, 2001), Japanese Patent Application No. 2008-519554 (filing date: Oct. 12, 2006) and Japanese Patent Application No. 2015-162578 (filing date: Aug. 20, 2015) are hereby incorporated in its entirety into the present specification by reference.

All documents, patent applications and technical standards described in the present specification are hereby incorporated into the present specification by reference to the same extent as if each of the individual document, patent application and technical standard were specifically and individually indicated to be incorporated by reference.

### INDUSTRIAL APPLICABILITY

The preparation to be applied to an ophthalmic device can be industrially utilized as a preparation for immersion, a preparation for coating agent, a preparation for preservation or a preparation for ultraviolet transmission inhibition for an ophthalmic device or, by coating the ophthalmic device with the preparation to be applied to an ophthalmic device, as a device for treating diseases of the ocular surface.

1. A method for preserving an ophthalmic device, the method comprising:
contacting the ophthalmic device with a preservation solution,
wherein the preservation solution comprises a compound in which a cinnamic acid ester having an amino group is covalently bonded to a carboxyl group of a glycosaminoglycan or a pharmaceutically acceptable salt thereof, and
wherein the compound has a degree of substitution ranging from 3 to 30%, where the degree of substitution is the ratio of the number of the covalently bonded cinnamic acid ester to the number of repeating disaccharide unit in the glycosaminoglycan or the pharmaceutically acceptable salt thereof.

2. The method according to claim 1, wherein the degree of substitution is from 3 to 20%.

3. The method according to claim 1, wherein the glycosaminoglycan is hyaluronic acid.

4. The method according to claim 1, wherein the cinnamic acid ester is covalently bonded to the carboxyl group via an amide bond.

5. The method according to claim 1, wherein the cinnamic acid ester is cinnamic acid aminoalkyl ester.

6. The method according to claim 5, wherein the number of carbon atoms in a main chain of an alkyl moiety of the cinnamic acid aminoalkyl ester is 2 or 3.

7. The method according to claim 1, wherein the compound is represented by the following general formula (I):
[Ar—CH—CH—COO—(CH₂)n-NH—]ₘ—HA′ (I)
wherein Ar represents a phenyl group which may have a substituent, n represents 2 or 3, HA′ represents a carboxy residue of the HA, m represents the ratio of amidated carboxyl groups among all carboxyl groups in the HA, which is 3 to 50% relative to the total number of carboxyl groups.
